(19) United States
(12) Patent Application Publication
(10) Pub. No.: US 2024/0215986 A1
(43) Pub. Date: Jul. 4, 2024

(54) JUNCTIONAL ASSISTANCE DEVICE FOR EXTREMITIES

(71) Applicant: Vitach Solutions, LLC, Indian Trail, NC (US)

(72) Inventors: Ian D. KNAPP, Indian Trail, NC (US); Zachary J. LEBLANC, Indian Trail, NC (US)

(21) Appl. No.: 18/147,397

(22) Filed: Dec. 28, 2022

(51) Int. Cl. A61B 17/132

(52) U.S. Cl. CPC A61B 17/1325

(57) ABSTRACT

A junctional assistance device designed to apply uniform pressure to a person in conjunction with a tourniquet. The junctional assistance device is a singular piece including a larger oval top surface that is lofted into a smaller oval shaped bottom surface. The junctional assistance device is designed to allow pressure to be transferred from the larger top surface to the smaller bottom surface.

### BACKGROUND

Junctional hemorrhage is a predominant cause of death in military operations. Particularly, injury and trauma to a person's pelvic region causes blood loss that cannot easily be stopped. This is due to the issue that the pelvic region has minimal surface areas that allows for hemostasis to be achieved while using a tourniquet. Current methods of treating injuries during military operations include various methods, one of which is using a tourniquet to apply pressure above the wounded region to restrict blood flow. Tourniquets are extremely helpful in stopping blood from flowing out of an open wound when properly used. The blood flow stoppage prolongs an injured person's life and prevents massive amounts of blood loss when used properly.

### FIELD OF THE INVENTION

The present invention is generally directed to junctional assistance devices, more specifically to junctional assistance devices for the iliac arteries.

### DESCRIPTION OF RELATED ART

Traditional tourniquets cannot always be practically applied to junctional wound areas, and hemostatic dressing is not always sufficient enough to achieve hemostasis. Additionally, modified junctional assistance devices can struggle to prevent blood flow without damaging or penetrating tissues. Some junctional assistance devices require the use of additional components (e.g., a balloon) to apply pressure to an injury. This is disadvantageous as operating (e.g., inflating) an additional component takes time and additional energy, which is not always possible due to an injury or while under fire during military operations. Current junctional tourniquets for the pelvic region use a plurality of separate components that are susceptible to breaking. When the components break, the tourniquet becomes unusable and increases the risk of not being able to stop the blood flow to a user's injury. Additionally, current junctional tourniquets require training and practice for proper use.

Yet another problem with current junctional tourniquet devices and methods of stopping blood flow is the amount time needed to stop blood flow to a wounded area. A person can bleed out within minutes depending on the severity of the injury. Tourniquets and other similar devices can take minutes to stop blood flow and/or apply pressure to a patient that results in severe pain. Therefore, there is a need for a junctional tourniquet device designed to rapidly decrease blood flow to injury while minimizing the amount of pressure and pain experienced by a patient.

### BRIEF SUMMARY

The subject matter described herein includes a junctional assistance device for extremities. The present invention includes a device designed to apply pressure to the inguinal and iliac arteries with a point of pressure being proximal to the inguinal ligament. The iliac arteries extend into the pelvic girdle distal to the aortic abdominal bifurcation. For example, and not limitation, the junctional assistance device is placed at the highest position of the iliac arteries within the pelvic girdle. In one embodiment, the junctional assistance device is meant to be used in conjunction with a tourniquet. In another embodiment, if a tourniquet is not available, the junctional assistance device is designed to allow the user to apply pressure by hand or compression wrap. In one embodiment, the junctional assistance device is a singular piece including a larger oval top surface that is lofted into a smaller oval shaped bottom surface. This allows for pressure to be transferred from a larger surface to a smaller surface. The smaller surface applies downward pressure atop the iliac artery with a rounded edge for more penetration through tissue and fat. The larger surface includes grooves to receive a tourniquet and to angle the tourniquet to increase the coverage of the iliac artery.

### BRIEF DESCRIPTION OF THE SEVERAL VIEWS OF THE DRAWINGS

The embodiments illustrated, described, and discussed herein are illustrative of the present invention. As these embodiments of the present invention are described with reference to illustrations, various modifications, or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

FIG. 1A illustrates an isometric view of a junctional assistance device according to one embodiment of the present invention.

FIG. 1B illustrates a front view of a junctional assistance device according to one embodiment of the present invention.

FIG. 1C illustrates a rear view of a junctional assistance device according to one embodiment of the present invention.

FIG. 1D illustrates a bottom view of a junctional assistance device according to one embodiment of the present invention.

FIG. 2A illustrates an isometric view of a junctional assistance device extension according to one embodiment of the present invention.

FIG. 2B illustrates a front view of a junctional assistance device extension according to one embodiment of the present invention.

FIG. 2C illustrates a side view of a junctional assistance device extension according to one embodiment of the present invention.

FIG. 2D illustrates a bottom view of a junctional assistance device extension according to one embodiment of the present invention.

FIG. 3A illustrates a top perspective view of a junctional assistance device according to one embodiment of the present invention.

FIG. 3B illustrates a front view of a junctional assistance device according to one embodiment of the present invention.

FIG. 3C illustrates a top view of a junctional assistance device according to one embodiment of the present invention.

FIG. 4 illustrates a junctional assistance device according to one embodiment of the present invention.

FIG. 5A illustrates an isometric perspective of a junctional assistance device according to one embodiment of the present invention.

FIG. 5B illustrates a side view of a junctional assistance device according to one embodiment of the present invention.

FIG. 5C illustrates a front view of a junctional assistance device according to one embodiment of the present invention.

FIG. 5D illustrates a top view of a junctional assistance device according to one embodiment of the present invention.

FIG. 5E illustrates a bottom view of a junctional assistance device according to one embodiment of the present invention.

FIG. 6 illustrates a method of using a junctional assistance device according to one embodiment of the present invention.

### DETAILED DESCRIPTION

For the purpose of promoting an understanding of the present disclosure, reference will be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “a composite” means at least one composite and can include more than one composite.

Throughout the specification, the terms “about” and/or “approximately” may be used in conjunction with numerical values and/or ranges. The term “about” is understood to mean those values near to a recited value. For example, “about 40 [units]” may mean within +/−25% of 40 (e.g., from 30 to 50), within +/−20%, +/−15%, +/−10%, +/−9%, +/−8%, +/−7%, +/−6%, +/−5%, +/−4%, +/−3%, +/−2%, +/−1%, less than +/−1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

As used herein, the verb “comprise” as is used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only”, and the like in connection with the recitation of claim elements, or the use of a “negative” limitation.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

The subject matter described herein includes a junctional assistance device for extremities. For example, and not limitation, the present invention includes a junctional assistance device designed to apply direct pressure to the inguinal arteries post-bifurcation of the descending aorta and before the artery descends posteriorly into the pelvic girdle. The junctional assistance device is meant to be used in conjunction with a tourniquet and/or if a tourniquet is not available, the junctional assistance device allows the user to apply pressure by hand or compression wrap. The junctional assistance device is a unitary piece in design and includes a larger top surface that is lofted into a smaller bottom surface. For example, and not limitation, the top surface and the bottom surface are a similar shape. In one embodiment, the present invention includes an oval-shaped top surface that is lofted into a smaller oval-shaped bottom surface. This allows for pressure to be transferred from a larger surface to a smaller surface. The smaller surface has a completely rounded edge to allow for the device to penetrate the tissue and fat and allow for more pressure to be applied downwards onto the iliac artery.

In one embodiment, the present invention includes a junctional assistance device for extremities including a top surface and a bottom surface, wherein the top surface includes at least one pressure area and at least one slot designed to receive a pressure component, and wherein the bottom surface is designed to uniformly apply pressure to an injured region of a person when pressure is applied to the top surface of the junctional assistance device. In another embodiment, the present invention further includes an extension component, wherein the extension component is designed to attach to the top surface of the junctional assistance device. The extension component further includes at least one pressure area, wherein the at least one pressure area is positioned to align with the at least one pressure area of the top surface. The extension component is removably attachable to the top surface, wherein the extension component is operable to attached to junctional assistance device when pressed onto the top surface. In another embodiment, the at least one pressure area is a polygonal shape. In one embodiment, the at least one pressure area is positioned on an end of the top surface. In yet another embodiment, the at least one pressure area includes at least two pressure areas. In yet another embodiment, the at least one pressure area of the at least two pressure areas is positioned at a first end of the top surface, wherein at least one pressure area is positioned at a second end of the top surface, wherein the first end and the second end are on opposite sides of the top surface. In one embodiment, the pressure component is a tourniquet. In yet another embodiment, the present invention includes at least one attachment component, wherein the attachment component is designed to connect to load-carrying equipment, wherein the load-carrying equipment includes a backpack, a helmet, a vest, and/or a satchel.

In another embodiment, the present invention includes a junctional assistance device for extremities including a top surface including at least two pressure areas and at least one slot designed to receive a pressure component, a bottom surface, wherein the at least two pressure areas include a first pressure area and a second pressure area, wherein the first pressure area is positioned on a first edge of the top surface, wherein the second pressure area is positioned on the second edge of the top surface, wherein the first edge is on an opposite side of the second edge, wherein the at least one slot is positioned between the first pressure area and the second pressure area, and wherein the bottom surface is designed to uniformly apply pressure to an injured region of a person when pressure is applied to the top surface of the junctional assistance device.

In one embodiment, the present invention includes a junctional assistance device further comprising an extension component, wherein the extension component is designed to attach to the top surface of the junctional assistance device. In another embodiment, the extension component further includes at least two pressure areas, wherein the at least two pressure areas of the extension component are positioned to align with the at least two pressure areas of the top surface. In yet another embodiment, the extension component is removably attachable to the top surface, wherein the extension component is operable to attach to the junctional assistance device when pressed onto the top surface. In one embodiment, the at least one pressure area is a polygonal shape. In another embodiment, each of the at least two pressure areas includes a top surface and a bottom surface, wherein the top surface is connected to the bottom surface via a sloped surface, wherein the top surface includes a smaller surface area than the bottom surface. In one embodiment, the at least two pressure areas of the extension component are the same shape as the at least two pressure areas of the junctional assistance device. In another embodiment, the junctional assistance device further comprises a second extension component, wherein the second extension component is designed to attach to the first extension component. In yet another embodiment, the pressure component includes a tourniquet. In one embodiment, the sloped surface includes an angle between about 120 degrees and about 125 degrees from the top surface of the extension.

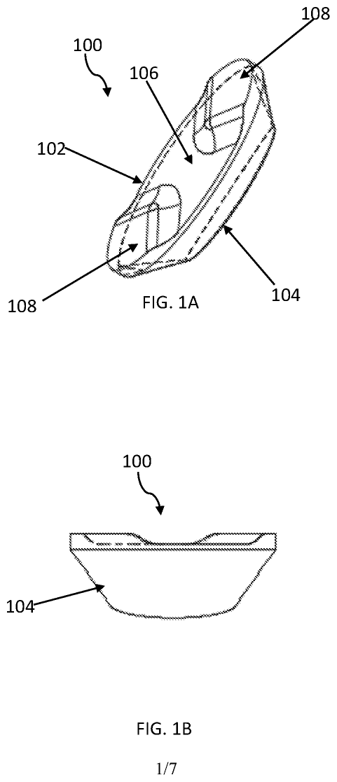

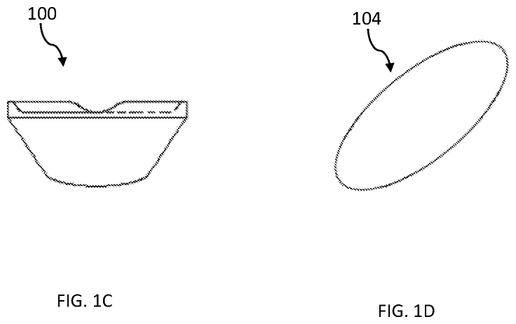

FIGS. 1A-1D illustrate a junctional assistance device according to one embodiment of the present invention. FIG. 1A illustrates a top perspective of a junctional assistance device according to one embodiment of the present invention. FIG. 1B illustrates a front view of a junctional assistance device. FIG. 1C illustrates a rear view of a junctional assistance device. FIG. 1D illustrates a bottom view of a junctional assistance device.

The junctional assistance device 100 includes a top surface 102 and a bottom surface 104. The top surface 102 is designed to receive a tourniquet along an opening 106 between two polygonal pressure areas 108. The bottom surface 104 is designed to generate uniform pressure on a person when pressure is applied to the top surface. In one embodiment, the top surface 102 and the bottom surface 104 are connected with a sloped surface. For example, and not limitation, the sloped surface has an angle between about 120 degrees and about 125 degrees relative to the bottom surface. The opening of the junctional assistance device 106 allows the tourniquet to lie across the junctional assistance device at an angle of about 48 degrees to allow the tourniquet to sit in parallel to the hip. Advantageously, the junctional assistance device matches the angle of the pelvic girdle.

In one embodiment, the top surface and the bottom surface are entirely flat and have an oval shape. The top surface has a larger surface area than the bottom surface. Alternatively, the top surface has a smaller surface area than the bottom surface. In yet another embodiment, the junctional assistance device includes a height extension. In another embodiment, the junctional assistance device includes a universal tourniquet holder, a rounded edge bottom, and a top surface lofted to a bottom surface at a pelvic girdle angle.

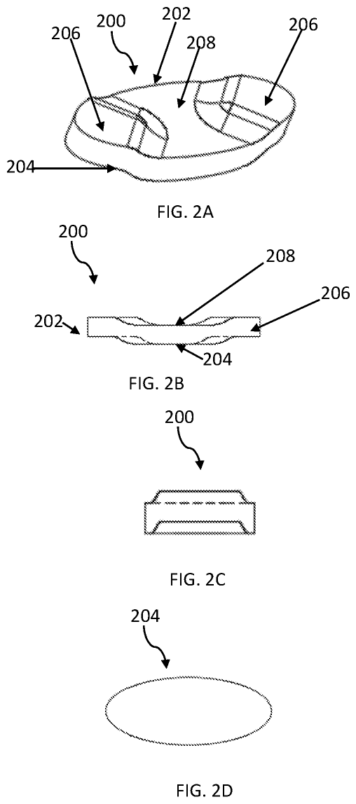

FIGS. 2A-2D illustrate a junctional assistance device extension according to one embodiment of the present invention. For example, and not limitation, the junctional assistance device is designed to be attached to a junctional assistance device as shown in FIGS. 1A-1D. FIG. 2A illustrates a top perspective view of a junctional assistance device extension. The junctional assistance device extension 200 includes a top surface 202 and a bottom surface 204. The top surface 202 includes a plurality of pressure areas 206 and a slot to receive a tourniquet 208. For example, and not limitation, the plurality of pressure areas includes a polygonal shape. For further example, and not limitation, the polygonal shape includes a trapezoidal shape, a square shape, a triangular shape, or a rectangular shape. In another embodiment, the pressure area includes a curved slope from a top surface of the pressure area to a bottom surface of the pressure area. FIG. 2B illustrates a front view of a junctional assistance device extension. FIG. 2C illustrates a side view of a junctional assistance device extension. FIG. 2D illustrates a bottom view of a junctional assistance device extension. Advantageously, the junctional assistance device extension increases the distance from a wound and/or trauma site and enables users of larger sizes to apply pressure to the intended wound site. In one embodiment, the junctional assistance device extension is designed to snap onto a junctional assistance device. In yet another embodiment, the junctional assistance device extension is designed to be pressed on a junctional assistance device.

In another embodiment, the bottom surface of the junctional assistance device extension is designed to match the lofted opening of the top of the junctional assistance device to allow for a press fit. The top of the junctional assistance device extension is a similar shape to the top of the junctional assistance device, which enables the junctional assistance device extension to perform identically to the junctional assistance device. The junctional assistance device extension allows for the top surface to sit at a greater height, thereby allowing for more downward pressure.

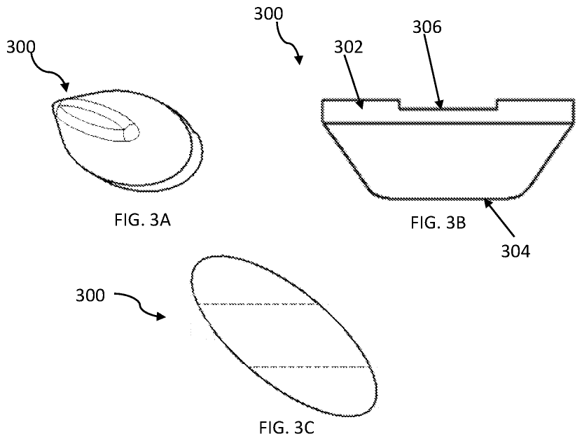

FIGS. 3A-3C illustrate a junctional assistance device according to one embodiment of the present invention. FIG. 3A illustrates an isometric perspective of a junctional assistance device. FIG. 3B illustrates a front view of a junctional assistance device. FIG. 3C illustrates a top view of a junctional assistance device. The junctional assistance device 300 includes a top surface 302 and a bottom surface 304. The top surface 302 includes a circular surface with a slot 306 to receive a tourniquet and other similar pressure applicators (e.g., a belt). Advantageously, the junctional assistance device is designed to decrease in size from the top surface to an entirely flat bottom surface. This enables the present invention to uniformly apply pressure to a body part. In one embodiment, the bottom surface is the same shape as the top surface. Alternatively, the bottom surface is a different shape than the top surface.

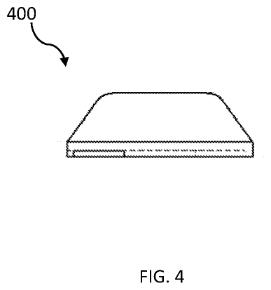

FIG. 4 illustrates another embodiment of a junctional assistance device including a small, rounded surface applying pressure to the junctional arteries and receiving pressure from a larger surface. The device is operable to receive a tourniquet on the larger surface that is extruded away from the body to allow for more pressure to the be applied from a tourniquet.

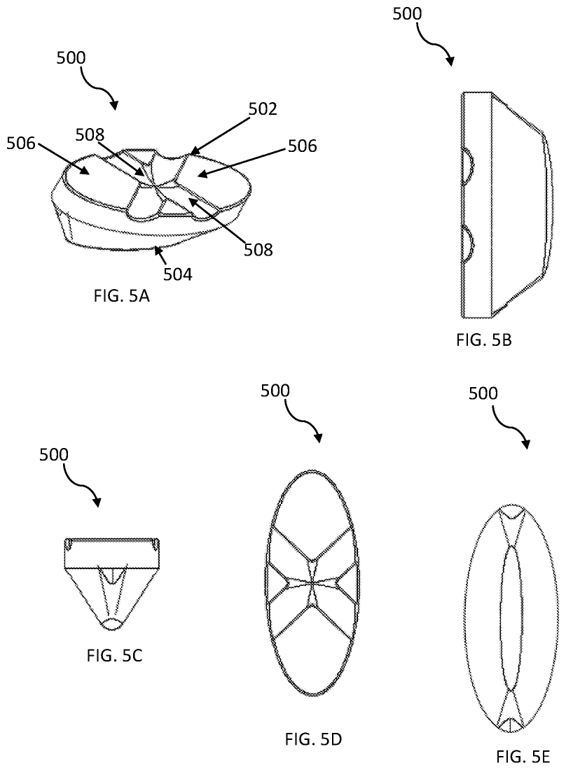

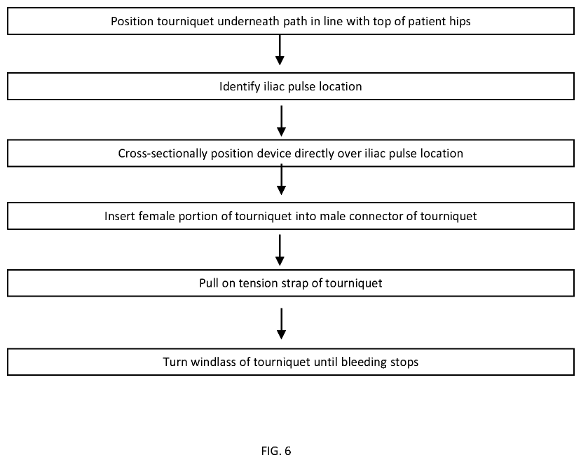

FIGS. 5A-5E illustrate a junctional assistance device according to one embodiment of the present invention. The junctional assistance device 500 includes a top surface 502 and a bottom surface 504. The top surface 502 includes a plurality of pressure areas 506 and a plurality of slots 508 to receive a tourniquet and/or other similar pressure applying devices. For example, and not limitation, the plurality of pressure areas includes a polygonal shape. For further example, and not limitation, the polygonal shape includes a trapezoidal shape, a square shape, a triangular shape, or a rectangular shape. As further example, the plurality of slots included a curved shape (e.g., half-pipe shaped) designed to improve the grip of the junctional assistance device with a tourniquet. In another embodiment, the pressure area includes a curved slope from a top surface of the pressure area to a bottom surface of the pressure area. The bottom surface 504 is smaller than the top surface to provide downward pressure onto a patient. In one embodiment, the bottom surface is a similar shape to the top surface. In another embodiment, the bottom surface is a circular shaped, a triangular shape, a rectangular shape, a trapezoidal shape, or other polygonal shapes. FIG. 5A illustrates an isometric view of the junctional assistance device 500 according to one embodiment of the present invention. FIG. 5B illustrates a side view of a junctional assistance device according to one embodiment of the present invention. FIG. 5C illustrates a front view of a junctional assistance device according to one embodiment of the present invention. FIG. 5D illustrates a top view of a junctional assistance device according to one embodiment of the present invention. FIG. 5E illustrates a bottom view of a junctional assistance device according to one embodiment of the present invention.

In one embodiment, the junctional assistance device is designed to be used to apply pressure to a junction area of a patient. For example, and not limitation, the junction area includes an upper extremity (e.g., arm), a lower extremity (e.g., foot), a pelvic region, a neck region, an abdomen region, and a thoracic region. Advantageously, the present invention is designed to be used with a tourniquet to apply pressure to an injured region.

The present invention is further operable to attach to and/or be stored in load-carrying equipment including but not limited to a backpack, a battery pack, a vest, a vehicle, and other load-carrying equipment via an attachment component. The attachment component includes a latch (e.g., snap latch), hook and loop tape, a clip, a retainer band, a pouch, and other similar attachment components.

In yet another embodiment, the present invention includes a method of preventing blood flow to at least one body part of a person. First, a tourniquet is positioned underneath a path in parallel with the top of a person's hips. Next, an iliac pulse location is identified, and the present invention is applied directly over the pulse location. For example, and not limitation, the junctional assistance device is positioned such that the junctional assistance device is perpendicularly positioned relative to the iliac artery near the inguinal ligament. Next, a female portion of the tourniquet is inserted into a male connector of a tourniquet windlass apparatus. Then, the tension strap of the tourniquet is pulled to provide tension over the present invention. Finally, the windlass of the tourniquet is turned until the bleeding has stopped.

### Example 1

| | Trial 1 | Trial 2 | Trial 3 | Trial 4 | Trial 5 |
|---|---|---|---|---|---|
| Patient 1 | 23 | 28 | 25 | 19 | 36 |
| Sex: F | seconds | seconds | seconds | seconds | seconds |
| Hip: 35 in | | | | | |
| Waist 24 in | | | | | |
| Patient 2 | 20 | 48 | 26 | 29 | 36 |
| Sex: M | seconds | seconds | seconds | seconds | seconds |
| Hip: 44 in | | | | | |
| Waist 38 in | | | | | |
| Patient 3 | 34 | N/A | 86 | 46 | 45 |
| Sex: M | seconds | | seconds | seconds | seconds |
| Hip: 44 in | | | | | |
| Waist 36 in | | | | | |
| Patient 4 | 36 | 34 | 49 | 29 | 19 |
| Sex: M | seconds | seconds | seconds | seconds | seconds |
| Hip: 42 in | | | | | |
| Waist 34 in | | | | | |
| Patient 5 | 46 | 48 | 52 | 24 | 26 |
| Sex: M | seconds | seconds | seconds | seconds | seconds |
| Hip: 42 in | | | | | |
| Waist 34 in | | | | | |

Table 1 illustrates the time it took to reach hemostasis while using a junctional assistance device according to one embodiment of the present invention on the iliac girdle of a patient. The junctional assistance device included a rounded bottom surface and matched a pelvic girdle angle. The patients who had medical experience performed better with device application with a positive correlation of approximately 0.58. This means that although medical experience and/or training increased the effectiveness of the junctional assistance device, medical experience and/or training was not required to effectively use the junctional assistance device. The mean average time was about 24 seconds (excluding a failure trial) to about 36 seconds until occlusion of a pulse was identified via a venous doppler system at the popliteal pulse, posterior tibial artery, and dorsalis pedis artery. Due to the different anatomy of each patient, prior identification and location of pulses were identified and marked. Doppler operator maintained constant analysis of pulse throughout the application of a junctional assistance device. Time was stopped at the clasping of the tourniquet windless, not at the cessation of the pulse. It was assumed that hemostasis was achieved after the clasping of the tourniquet windless. Confirmation of a successful device use was done by checking palpation of the pedal pulses of the patient via hand and with a veinous doppler device.

Too many height extensions can result in a capsized appearance of the junctional assistance device while positioned on a patient. It was further noticed that the junctional assistance device would slide or straighten out, resulting in the junctional assistance device being positioned in parallel to the iliofemoral artery junction. Although this still allowed cessation of blood flow, it introduced a greater degree of human error. To mitigate this, the junctional assistance device was modified to include a flattened side edge, thereby reducing the lean of the device both while under tension and alone atop the patient skin.

In one embodiment, the present invention further includes a lateral extension. In yet another embodiment, the junctional assistance device is designed to match other junctional areas (e.g., armpit, neck). In another embodiment, the present invention further includes a plurality of slits to receive a plurality of straps. In yet another embodiment, the junctional assistance device includes a doppler component. The doppler component is designed to monitor and capture blood flow data corresponding to a patient. For example, and not limitation, in one embodiment, the doppler component is attached to a surface of the junctional assistance device. In another embodiment, the doppler component is positioned on the interior of the junctional assistance device. In yet another embodiment, the doppler device is a flush mount doppler device.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A junctional assistance device for extremities comprising:
a top surface; and
a bottom surface;
wherein the top surface includes at least one pressure area and at least one slot designed to receive a pressure component;
wherein the bottom surface is designed to uniformly apply pressure to an injured region of a person when pressure is applied to the top surface of the junctional assistance device.

2. The junctional assistance device of claim 1, further comprising an extension component, wherein the extension component is designed to attach to the top surface of the junctional assistance device.

3. The junctional assistance device of claim 2, wherein the extension component further includes at least one pressure area, wherein the at least one pressure area of the extension component is positioned to align with the at least one pressure area of the top surface.

4. The junctional assistance device of claim 2, wherein the extension component is removably attachable to the top surface of the junctional assistance device, wherein the extension component is designed to attach to the top surface of the junctional assistance device when pressed onto the top surface.

5. The junctional assistance device of claim 1, wherein the at least one pressure area is a polygonal shape.

6. The junctional assistance device of claim 1, wherein the at least one pressure area is positioned on an end of the top surface.

7. The junctional assistance device of claim 1, wherein the at least one pressure area includes at least two pressure areas.

8. The junctional assistance device of claim 7, wherein at least one pressure area of the at least two pressure areas is positioned at a first end of the top surface, wherein a different at least one pressure area is positioned at a second end of the top surface, wherein the first end and the second end are on opposite sides of the top surface.

9. The junctional assistance device of claim 1, wherein the pressure component is a tourniquet.

10. The junctional assistance device of claim 1 further including at least one attachment component, wherein the attachment component is designed to connect to load carrying equipment, wherein the load carrying equipment includes a backpack, a helmet, a vest, and/or a satchel.

11. A junctional assistance device for extremities comprising:
a top surface including at least two pressure areas and at least one slot designed to receive a pressure component; and
a bottom surface;
wherein the at least two pressure areas include a first pressure area and a second pressure area;
wherein the first pressure area is positioned on a first edge of the top surface;
wherein the second pressure area is positioned on a second edge of the top surface;
wherein the first edge is on an opposite side of the second edge;
wherein the at least one slot is positioned between the first pressure area and the second pressure area; and
wherein the bottom surface is designed to uniformly apply pressure to an injured region of a person when pressure is applied to the top surface of the junctional assistance device.

12. The junctional assistance device of claim 11, further comprising an extension component, wherein the extension component is designed to attach to the top surface of the junctional assistance device.

13. The junctional assistance device of claim 12, wherein the extension component further includes at least two pressure areas, wherein the at least two pressure area of the extension component is positioned to align with the at least two pressure areas of the top surface.

14. The junctional assistance device of claim 12, wherein the extension component is removably attachable to the top surface of the junctional assistance device, wherein the extension component is operable to attach to the top surface of the junctional assistance device when pressed onto the top surface.

15. The junctional assistance device of claim 11, wherein the at least two pressure areas are a polygonal shape.

16. The junctional assistance device of claim 11, wherein each of the at least two pressure areas include a top surface and a bottom surface, wherein the pressure area top surface is connected to the pressure area bottom surface via a curved surface, wherein the top surface includes a smaller surface area than the bottom surface.

17. The junctional assistance device of claim 13, wherein the at least two pressure areas of the extension component are a same shape as the at least two pressure areas of the junctional assistance device.

18. The junctional assistance device of claim 12, wherein the extension component further comprises a first extension component and a second extension component, wherein the second extension component is designed to attach to the first extension component.

19. The junctional assistance device of claim 12, wherein the pressure component includes a tourniquet.

20. The junctional assistance device of claim 11, wherein the top surface of the junctional assistance device is connected to the bottom surface of the junctional assistance device via a sloped surface, wherein the sloped surface includes an angle between about 120 degrees and about 125 degrees from the top surface of the extension component.
